(19) United States
(12) Patent Application Publication
(10) Pub. No.: US 2019/0329032 A1
(43) Pub. Date: Oct. 31, 2019

(54) NEUROMODULATION LEAD FOR REDUCING INTERACTIONS WITH MRI

(71) Applicant: BIOTRONIK SE & CO. KG, BERLIN (DE)

(72) Inventors: JEFFREY A. VON ARX, LAKE OSWEGO, OR (US); ROBERT R. SHURIG, TUALATIN, OR (US); KRISHNA K.N. KURPAD, PORTLAND, OR (US); BRIAN P. SUTTON, WEST LINN, OR (US); JAMES E. BROWN, TIGARD, OR (US); PAUL STADNIK, LAKE OSWEGO, OR (US)

(21) Appl. No.: 16/396,981

(22) Filed: Apr. 29, 2019

Related U.S. Application Data
(60) Provisional application No. 62/663,286, filed on Apr. 27, 2018.

(51) Int. Cl. A61N 1/08; H01B 13/06; H01B 7/00; H01B 7/02; H01F 27/28; A61N 1/05

(52) U.S. Cl. CPC A61N 1/086; H01B 13/06; A61N 1/0551; H01B 7/0291; H01F 27/2828; H01B 7/0009

(57) ABSTRACT

A lead has at least a first and a second conductor. The first conductor and the second conductor each have an electrically conducting core that is surrounded by an electrical insulator. The electrical insulator of the first conductor if formed from a first material, and the electrical insulator of the second conductor is formed from a second material. The first material differs from the second material.

### CROSS-REFERENCE TO RELATED APPLICATION

This application claims the priority, under 35 U.S.C. § 119(e), of provisional application No. 62/663,286 filed Apr. 27, 2018; the prior application is herewith incorporated by reference in its entirety.

### BACKGROUND OF THE INVENTION

### Field of the Invention

Conventional designs of percutaneous SCS leads are in general not suitable for MR-Conditional Full Body Scan (FBS) labeling.

Magnetic resonance imaging (MRI) devices put out very large radio frequency (RF) fields during operation. The energy in these fields is picked up by conductors in the SCS lead. If the leads do not have mechanisms for dissipating this energy, then the energy reaches the electrodes of the lead where it is dissipated as heat. Disadvantageously, such an electrode heating can damage tissue of the patient.

U.S. Pat. No. 9,399,129 describes a medical lead having multiple conductors which are wound in an interleaved manner. Furthermore, U.S. patent publication No. 2016/0331960 A1 describes different materials suited as electrical insulation for leads.

Based on the above, there is a desire for providing comparatively simple leads, particularly SCS leads, which do not suffer from electrode heating when subjected to MRI, and which are particularly MR conditional Full Body Scan.

### SUMMARY OF THE INVENTION

This objective is solved by a lead having the features of the independent claim. Particular embodiments of this aspect of the present invention are stated in the corresponding sub claims.

According to the independent claim, a lead for transporting an electrical current is disclosed, comprising at least a first and a second conductor. The first conductor and the second conductor each comprise an electrically conducting core (comprising e.g. at least one wire or several wires) that is surrounded by an electrical insulator. The electrical insulator of the first conductor consists of a first material, and the electrical insulator of the second conductor consists of a second material, where the first material differs from the second material.

The respective core can e.g. be formed by a single wire or a plurality of adjacent wires.

Advantageously, by using different materials for the electrical insulations, the present invention allows to tune (e.g. during the design phase) of each conductor's resonant frequency to e.g. ensure that none of the conductors are in resonance at the used or any useable MR frequencies depending on Lamor frequencies. Particular, in this way, the present invention allows too tune the individual conductor such that MR-Conditional FBS labeling is enabled.

According to an embodiment of the present invention, the first material and the second material are chosen such that the lead comprises a resonance frequency that is different from a frequency used in an MRI device, particularly different from 64 MHz (e.g. for 1.5 T MRI devices) or different from 128 MHz (e.g. for 3T MRI devices).

Furthermore, according to an embodiment of the present invention, the first material and the second material are selected from the group of the following materials: a ceramics, a polymer, ETFE, PFA, PTFE, polyimide, aluminum oxide, barium titanate, and titanium dioxide.

Furthermore, according to an embodiment of the present invention, the conductors of the lead, e.g. the first and the second conductor, each form a helical coil.

According to a further embodiment, the respective conductor can also extend linearly. Furthermore, alternatively, two or more conductors of the lead can be braided or wound in an interleaved manner.

Further, according to an embodiment of the present invention, the lead comprises a plurality of conductors including the first and the second conductor. Each conductor of the plurality of conductors other than the first and the second conductor also comprises an electrically conducting core (e.g. at least one wire or several wires) that is surrounded by an electrical insulator formed out of the first material or out of the second material, or out of a further material different from the first and the second material.

Furthermore, according to an embodiment of the present invention, the plurality of conductors may consist of at least eight, or at least 16 conductors.

Furthermore, according to an embodiment of the present invention, the lead comprises a lead body insulator surrounding each individual conductor. The lead body insulator can be formed out of or can comprise polyurethane or silicone.

Furthermore, according to an embodiment of the present invention, the lead comprises at least one further conductor formed by a non-insulated (i.e. bare) electrically conducting member, which is electrically insulated with respect to its surrounding by electrical insulators of adjacent conductors of the lead and/or by the lead body insulator. Particularly that electrically conducting member may be formed by at least one or several wires.

Furthermore, according to an embodiment of the present invention, the first and the second conductor are co-radial and/or co-axial helical coils. Besides the first and second conductor also each remaining conductor (see above) can be a co-radial and/or a co-axial helical coil with respect to another conductor of the lead.

Particularly, two adjacent conductors form co-radial helical coils if they are arranged one inside the other and have the same radius and pitch. Further, particularly, two co-axial helical coils (or conductors) are co-axial in case they have a common coil axis around which the respective coil (or conductor) is wound.

Furthermore, according to an embodiment of the present invention, the conductors (e.g. the first and the second conductor and particularly the remaining conductors if present) form an inner coil structure and a co-axial outer coil structure surrounding the inner coil structure. Each coil structure comprises a plurality of conductors and each conductor forms a helical coil, wherein particularly the helical coils (conductors) of the inner coil structure are co-radial and/or wherein the helical coils (conductors) of the outer coil structure are co-radial.

Also here, particularly, two coil structures are co-axial in case they have a common coil axis around which the conductors of the respective coil structure are wound.

Furthermore, according to an embodiment of the present invention, the lead is a medical lead (i.e. a lead of a medical device). Particularly, the lead is an implantable medical lead.

Furthermore, according to an embodiment of the present invention, the lead is an electrode lead comprising a plurality of electrodes. Each electrode may form a section, particularly a circumferential section, of an outer surface of the lead, wherein each electrode is electrically connected to one of the conductors. Particularly the lead comprises at least two electrodes, particularly at least eight electrodes, particularly at least 16 electrodes.

Furthermore, according to an embodiment of the present invention, the lead is an SCS lead, i.e. a lead adapted for spinal cord stimulation (SCS).

According to a further aspect of the present invention, a method for producing a lead, particularly a lead according to the present invention, is disclosed. At least a first and a second conductor are provided, wherein the first conductor and the second conductor each comprise an electrically conducting core (see e.g. above) that is surrounded by an electrical insulator. The electrical insulator of the first conductor is made out of a first material, the electrical insulator of the second conductor is made out of a second material, and the first material differs from the second material. The conductors are surrounded by an outer lead body insulator.

The materials that can be used are already stated above.

Particularly, according to an embodiment of the method according to the present invention, the first material and the second material are chosen such that the respective conductor comprises a resonance frequency that is different from a pre-defined frequency used in an MRI device, particularly different from 64 MHz (e.g. for 1.5 T MRI devices) or different from 128 MHz (e.g. for 3T MRI devices), see also above.

Particularly, the invention allows providing a MR Conditional full body scan SCS lead which is less complex than known solutions. Particularly, the invention allows for shifting the resonant frequency of each conductor in the lead to ensure all conductors avoid resonance at MR frequencies.

Other features which are considered as characteristic for the invention are set forth in the appended claims.

Although the invention is illustrated and described herein as embodied in a neuromodulation lead for reducing interactions with MRI, it is nevertheless not intended to be limited to the details shown, since various modifications and structural changes may be made therein without departing from the spirit of the invention and within the scope and range of equivalents of the claims.

The construction and method of operation of the invention, however, together with additional objects and advantages thereof will be best understood from the following description of specific embodiments when read in connection with the accompanying drawings.

### BRIEF DESCRIPTION OF THE SEVERAL VIEWS OF THE DRAWING

FIG. 1A is a diagrammatic, side view of a lead comprising co-radial conductors having electrical insulations, wherein each insulation can consist of one of at least two different materials;

FIG. 1B is an enlarged, partially cut-away sectional view of a section of the lead shown in FIG. 1A;

FIG. 1C is a sectional view of a conductor shown in FIG. 1B; and

FIG. 2 is a partially cut-away view showing the lead comprising an outer and a co-axial an inner coil structure, each structure comprising a plurality of co-radial conductors having electrical insulations, wherein each insulation can consist of one of at least two different materials.

### DETAILED DESCRIPTION OF THE INVENTION

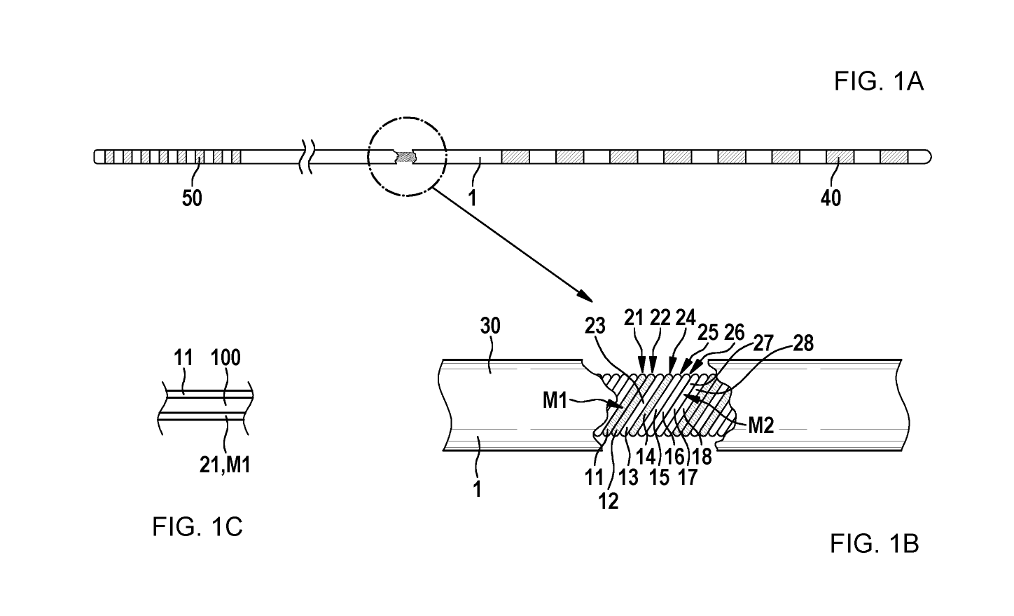

Referring now to the figures of the drawings in detail and first, particularly to FIGS. 1A-1C thereof, there is shown a lead 1 according to the present invention which comprises at least a first and a second conductor 12, 13, wherein the first conductor 12 and the second conductor 13 each comprise an electrically conducting core 100 that is surrounded by an electrical insulator 21, 22, 23 (as shown in FIG. 1C), wherein the electrical insulator 22 of the first conductor 12 consists of a first material M1, and wherein—analogously—the electrical insulator 23 of the second conductor 13 consists of a second material M2, wherein the first material M1 differs from the second material M2.

Furthermore, the lead 1 can comprises a lead body insulator 30 surrounding each individual conductor 12, 13 for providing further insulation and protection of the single conductors 12, 13.

Now, in order to avoid RF heating during MRI, conductors 11, 12, . . . , 18 of lead 1 need inductance L and capacitance C. One way to incorporate inductance and capacitance into the lead 1 is to coil the conductors 11, 12 along the length of the lead 1 as shown in FIG. 1B, which turns each conductor 11, 12 into a coil (or solenoid structure) with winding to winding parasitic capacitance. The LC combination creates a filter that blocks the high frequency MRI induced RF signals, while allowing the low frequency biological signals to pass unimpeded. Most SCS leads available today use straight conductors, which have very little inductance.

Particularly, the lead shown in FIG. 1A can be a co-radial SCS lead 1 with 8 electrodes 40 on a distal end of the lead 1, wherein each of the electrodes 40 is connected via one of the conductors 11, 18 to a connector contact 50 arranged on the proximal end of the lead 1. FIG. 1B shows the coiled conductors 11, 18. In FIG. 1B the three conductors 11, 12 and 14 are coated with an electrical insulator 21, 22, 24 consisting of a first material M1, while the five remaining conductors 13, 15, 16, 17, 18 are coated with an electrical insulator 23, 25, 26, 27, 28 consisting of a second material M2 that differs from the first material M1.

In addition to adding inductance and capacitance, resonance at MRI RF frequencies needs to be avoided in leads designed for MRI labeling. RF frequencies for MRI devices are e.g. ˜64 MHz for 1.5 T machines, and ˜128 MHz for 3T machines. If a conductor 11, 12 on the lead 1 shown in FIG. 1B resonates at MRI RF frequencies then the impedance of the lead 1 drops dramatically at these frequencies and very little energy is dissipated in the lead 1. The result is much more MRI induced current flowing through the lead 1 which results in much more RF heating at the electrodes 40. Avoiding resonance is particularly challenging for leads 1, particularly SCS leads 1, which typically have 8 different conductors 11, 18 connected to 8 different electrodes 40. Each conductor 11, 18 has its own resonance frequency which is distinct from the resonance frequency of the other conductors because each electrode 40 is typically located at a different distance along the lead 1, and hence the conductor 11, 18 connected to each electrode 40 has a different length. So, an 8 conductor lead 1 is much more challenging to design to avoid resonance than a typical 2 conductor cardiac lead because there are 4 times as many conductors that need to be configured to avoid resonance. Worse yet, some SCS leads incorporate paddle electrode arrays with 16 or more separate conductors connecting 16 or more different electrodes. These large number of electrodes 30 multiply the odds that at least one conductor 11, 18 connected to one electrode 40 will be in resonance at MRI frequencies.

To avoid such resonance frequencies, an embodiment of the present invention particularly uses coiled conductors 11, 18 for the lead 1, particularly an SCS lead 1, with at least two different materials M1, M2 as electrical insulator 21, 28 on the individual conductor core/wires 100 as shown e.g. in FIG. 1B. The coiled conductors 11, 18 give inductance because the coiling creates a solenoid structure, and the coiling also gives capacitance due to increased conductor length, which is capacitive coupled to neighboring windings. The different insulators 21, 28 allow each conductor's self-resonance to be selected during the design phase to avoid MRI frequencies. Some conductors have material M1 as insulator material and some conductors have material M2 as insulator material. The inductance of a solenoid is approximated by the well-known equation

$$L = {μ}_{0} \frac{{N}^{2} A}{l} ,$$

the capacitance is approximated by

$$C = \frac{{ɛ}_{0} {ϵ}_{r} A}{d} ,$$

and the resonance frequency is approximated by

$$f = \frac{1}{2 π \sqrt{LC}} .$$

The dielectric constant εᵣ is a function of the insulation material M1, M2 surrounding the respective conductor 11, 18. By changing the insulation material εᵣ changes, and hence the resonance frequency changes for the respective conductor M1, M2. The preferred materials M1, M2 for insulating the individual lead conductors 11, 18 are e.g. ETFE, PFA, and PTFE. The dielectric constant εᵣ for these materials are shown in Table 1 below. As can be seen ETFE, has a dielectric constant about 25% greater than PTFE or PFA. Therefore, in one embodiment ETFE is used on at least one conductor 11, 18, while PFA or PTFE are used on the other conductors.

TABLE 1

| | ETFE | PFA | PTFE |
|---|---|---|---|
| Dielectric Constant | 2.6 | 2.1 | 2.1 |

In one embodiment 8 different conductors connected to 8 different electrodes are all wound co-radially as shown in FIG. 1B.

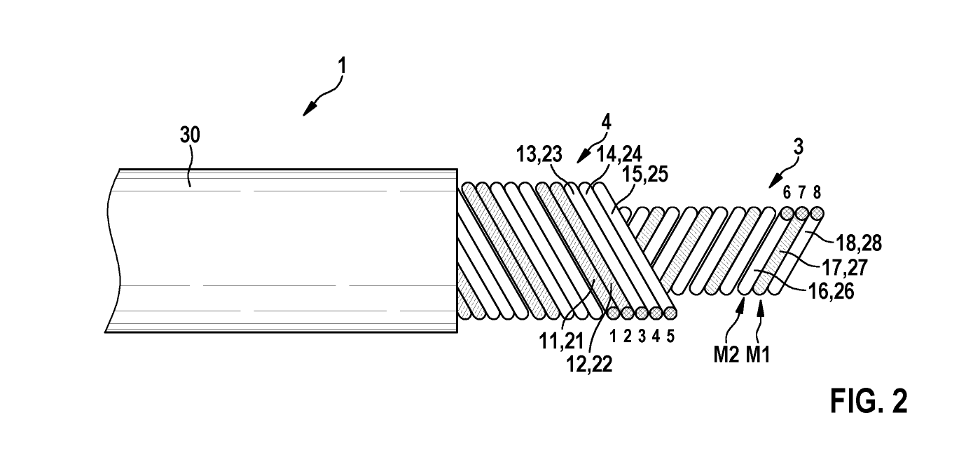

FIG. 2 shows a further embodiment of the lead 1 according to the present invention also using two different insulator materials M1, M2. Here, two conductors 11, 12 of an outer coil 4 structure comprised of 5 conductors 11, 12, 13, 14, 15 in form of co-radial helical coils comprise electrical insulators 21, 22 formed out of a first material M1. Further, the lead comprises an inner coil structure 3 surrounded by the co-axial outer coil structure 4, wherein the inner coil structure 3 is comprised of three conductors 16, 17, 18 in form of co-radial helical coils, wherein one conductor 17 is formed out of the first Material M1. The other conductors 13, 14, 15, and 16, 18 comprise insulators 23, 24, 25, 26, 28 made out of a different second material M2.

Here, in the second embodiment, a co-axial/co-radial design is used for the conductors 11, 18. In this embodiment there are two (or even more) layers of coiled conductors, namely an inner layer or coil structure 3 and an outer layer (or coil structure) 4. This construction is more complicated than the co-radial design shown in FIGS. 1A-1C, but it has several advantages. First, the outer coil 4 has less conductors than the co-radial design shown in FIGS. 1A-1C (since some of the conductors are moved to the inner coil 3), and less conductors means that each conductor has a finer pitch. The finer pitch results in a much greater inductance (inductance is proportional to the square of the pitch), which results in a better MRI induce RF current rejection. Second, the conductors 16, 18 in the inner coil structure 3 are shielded by the outer coil structure 4. Third, there is greater average capacitance between conductors in this design since the average distance between conductors 11, 18 is reduced. The reduced average distance is because of the finer pitch of both the inner and outer coil structure 3, 4 compared to the co-radial coil of FIGS. 1A-1C, and the nested arrangement of the two coil structures 3, 4 which causes coupling between the coils structures 3, 4. The greater average capacitance results in a better (lower cut off frequency) low pass (LC) filter. In addition, the inductive and capacitive coupling mechanisms result in more current sharing between the conductors at MRI frequencies. This helps average out the MRI induced currents on each conductor, eliminating peaks. One example implementation of this co-axial/co-radial design shown in FIG. 2 is detailed in Table 2 below.

In another embodiment at least one conductor (e.g. of the conductors 11, 18 shown in FIG. 1A-1C or 2) has no separate insulation around it at all (insulation to the body 30 is still provided by the lead body insulator/tube 30 which is typically silicone or polyurethane. This embodiment not only changes the resonance frequency of the conductor(s) with no separate insulation, but also allows all the conductors 11, 18 to be packaged closer together, resulting in a tighter winding pitch and higher overall inductance for each conductor. Particularly, at most, every other conductor can be without insulation.

Particularly, having different insulation materials M1, M2 on different filars of the inner and/or outer coil structure allows fine tuning of the resonant frequency of the lead 1 for each electrode 40. If a certain lead length leads to one or more electrodes 40 being in electrical resonance at MRI frequencies, then the insulation material M1, M2 can be changed on that particular filar/conductor to shift the resonance away from MRI frequencies. Furthermore, the present invention allows for visual identification between conductors during the manufacturing process (e.g. to make sure that the appropriate conductor gets welded to the appropriate contact). Particularly, colorants can be added to one or more of the coatings to make it readily apparent which conductor is which. Particularly, in the above-described embodiment in which the insulation is removed altogether on one or more filars/conductors, the pitch can be increased which increases the inductance and improves MRI performance. In one embodiment of this, only every other conductor is insulated.

It will be apparent to those skilled in the art that numerous modifications and variations of the described examples and embodiments are possible in light of the above teaching. The disclosed examples and embodiments are presented for purposes of illustration only. Therefore, it is the intent to cover all such modifications and alternate embodiments as may come within the true scope of this invention.

1. A lead, comprising:
at least a first conductor and a second conductor, said first conductor and said second conductor each having an electrically conducting core and an electrical insulator surrounding said electrically conducting core, said electrical insulator of said first conductor formed of a first material, and said electrical insulator of said second conductor formed of a second material, wherein said first material differing from said second material.

2. The lead according to claim 1, wherein said first material and said second material are chosen such that said first and second conductors respectively comprise a resonance frequency that is different from a frequency used in an magnetic resonance imaging device.

3. The lead according to claim 1, wherein said first material and said second material are selected from the group consisting of: ceramics, polymers, ETFE, PFA, PTFE, polyimides, aluminum oxides, barium titanate, and titanium dioxide.

4. The lead according to claim 1, wherein said first and the second conductors each form a helical coil.

5. The lead according to claim 1, wherein said first and second conductors are two of a plurality of conductors, each of said conductors other than said first and second conductors has said electrically conducting core and a further electrical insulator surrounding said electrically conducting core, said further electrical insulator formed from a material selected from the group consisting of said first material, said second material, and a further material being different from said first and second materials.

6. The lead according to claim 5, further comprising a lead body insulator surrounding each of said conductors.

7. The lead according to claim 6, further comprising at least one further conductor disposed with said plurality of conductors, said at least one further conductor is formed as a non-insulated electrically conducting member, which is electrically insulated with respect to its surrounding by means of said electrical insulator and said further electrical insulator of adjacent ones of said conductors and/or by means of said lead body insulator.

8. The lead according to claim 1, wherein said first and second conductors are co-radial and/or co-axial helical coils.

9. The lead according to claim 5, wherein said plurality of conductors form an inner coil structure and a co-axial outer coil structure surrounding said inner coil structure, wherein each of said inner coil structure and said co-axial outer coil structure contain at least one of said conductors of said plurality of conductors.

10. The lead according to claim 1, wherein the lead is a medical lead.

11. The lead according to claim 1, wherein the lead is an electrode lead having a plurality of electrodes.

12. The lead according to claim 1, wherein the lead is adapted for spinal cord stimulation.

13. A method for producing a lead, which comprises the steps of:
providing at least a first and a second conductor, the first conductor and the second conductor each having an electrically conducting core and an electrical insulator surrounding said electrically conducting core;
forming the electrical insulator of the first conductor out of a first material;
forming the electrical insulator of the second conductor out of a second material, wherein the first material differing from the second material; and
surrounding the first and second conductors with an outer lead body insulator.

14. The method according to claim 13, wherein the first material and the second material are chosen such that the first and second conductors comprise a resonance frequency that is different from a given frequency used in a magnetic resonance imaging device.
